(19) United States
(12) Patent Application Publication
(10) Pub. No.: US 2003/0054185 A1
(43) Pub. Date: Mar. 20, 2003

(54) Biocidal controlled-release formulations

(75) Inventors: Peter Ottersbach, Windeck (DE); Beate Kossman, Hagen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: CREAVIS GESELLSCHAFT F. TECHN.U.INNOVATION MBH, Marl (DE)

(21) Appl. No.: 10/244,163

(22) Filed: Sep. 16, 2002

(30) Foreign Application Priority Data
Sep. 14, 2001 (DE) 101 45 529.1

(51) Int. Cl. C12N 9/64; C12N 9/78

(52) U.S. Cl. 428/474.4; 428/500; 428/523; 435/226; 435/227

(57) ABSTRACT

The present invention provides an antimicrobial, controlled-release composition, which includes:

at least one antimicrobial polymer comprising at least one polymerized monomer unit selected from the group including:

2-tert-butylaminoethyl methacrylate, 2-diethylaminoethyl methacrylate, 2-diethylaminomethyl methacrylate, 2-tert-butylaminoethyl acrylate, 3-dimethylaminopropyl acrylate, 2-diethylaminoethyl acrylate, 2-dimethylaminoethyl acrylate, dimethylaminopropylmethacrylamide, diethylaminopropylmethacrylamide, N-3-dimethylaminopropylacrylamide, 2-methacryloyloxyethyltrimethylammonium methosulfate, 2-diethylaminoethyl methacrylate, 2-methacryloyloxyethyltrimethylammonium chloride, 3-methacryloylaminopropyltrimethylammonium chloride, 2-methacryloyloxyethyltrimethylammonium chloride, 2-acryloyloxyethyl-4-benzoyldimethylammonium bromide, 2-methacryloyloxyethyl-4-benzoyldimethylammonium bromide, allyltriphenylphosphonium bromide, allyltriphenylphosphonium chloride, 2-acrylamido-2-methyl-1-propanesulfonic acid, 2-diethylaminoethyl vinyl ether, 3-aminopropyl vinyl ether, and combinations thereof; and

at least 0.5% by weight of at least one organic solvent. The present invention also provides for methods of making and using the composition.

### BACKGROUND OF THE INVENTION

1. Field of the Invention

The invention relates to biocidal controlled-release formulations made from antimicrobial polymers, a process for preparing the controlled-release formulations, and their use.

2. Discussion of the Background Art

It is highly undesirable for bacteria to become established or to spread on the surfaces of piping, on containers and on packaging. Slime layers frequently form and permit sharp rises in microbial populations, and these can lead to persistent impairment of the quality of water, drinks, or foods, to spoilage of the product and harm to the health of consumers.

Bacteria must be kept away from all areas of life in which hygiene is important. This includes textiles for direct body contact, especially in the genital area, or for the care of the elderly and sick. Bacteria must also be kept away from surfaces of furniture and instruments used in patient-care areas, especially in areas for intensive care or neonatal care, and in hospitals, especially in the areas where medical intervention takes place, and in isolation wards for critical cases of infection, and also in toilets.

A current method for treating equipment, the surfaces of furniture or of textiles, to resist bacteria either when necessary or else as a precautionary measure, is to use chemicals, solutions or mixtures thereof, which are disinfectants and thus have fairly broad general antimicrobial action. Chemical agents of this type act nonspecifically and are frequently themselves toxic or irritating, or form degradation products which are hazardous to health. In addition, people frequently exhibit intolerance to these materials once they have become sensitized.

Another method of counteracting the surface spread of bacteria is to incorporate substances with an antimicrobial action into a matrix.

Another challenge of constantly increasing significance is the elimination of algal growth on surfaces, since there are now many external surfaces of buildings with plastic cladding. Plastic cladding is particularly susceptible to colonization by algae. In addition to giving an undesirable appearance, this algae colonization can in some circumstances also impair the functioning of the components concerned. One relevant example is the colonization by algae of surfaces with a photovoltaic function.

Another form of microbial contamination for which no technically satisfactory solution has been found is the fungal infestation of surfaces. For example, Aspergillus niger infestation of joints or walls in wet areas within buildings not only impairs appearance but also has serious health implications, since many people are allergic to the substances given off by the fungi. This can result in serious chronic respiratory disease.

In the marine sector, the fouling of boats' hulls affects costs, since the growth of fouling organisms is attended by an increase in the boats' flow resistance and thus a marked increase in fuel consumption. Problems of this type have hitherto generally been countered by incorporating toxic heavy metals or other low-molecular-weight biocides into antifouling coatings, with the aim of mitigating the problems described. To this end, the damaging side effects of coatings of this type are accepted, but as society's environmental awareness rises this state of affairs is increasingly problematic.

U.S. Pat. No. 4,532,269, for example, discloses a terpolymer made from butyl methacrylate, tributyltin methacrylate, and tert-butylaminoethyl methacrylate. This copolymer is used as an antimicrobial paint for ships, and the hydrophilic tert-butylaminoethyl methacrylate promotes slow erosion of the polymer, thus releasing the highly toxic tributyltin methacrylate as active antimicrobial ingredient. Here, the copolymer prepared with aminomethacrylates is merely a matrix or carrier for the microbicidal ingredients, which can diffuse or migrate out of the carrier material. Sooner or later, polymers of this type lose their activity, once the necessary minimum inhibitor concentration (MIC) is no longer achieved at the surface.

European patent application EP 0,862,858 also discloses that copolymers of tert-butylaminoethyl methacrylate, a methacrylate with a secondary amino function, have microbicidal properties. Systems developed in the future will also have to be based on novel compositions with improved effectiveness if undesirable resistance phenomena in the microbes are to be avoided, particularly bearing in mind the microbial resistance known from antibiotics research.

Under some circumstances it might also be required to use other biocidal substances alongside the formulations which are purely contact-microbicidal. One situation in which this is appropriate is when the systems to be freed from microbes are flow-through systems in which it is impossible to ensure complete and sufficient contact with the microbially contaminated water. Although in principle it is possible to add conventional low-molecular-weight biocides, the above-described concerns relating to environmental toxicology mean that this is not advisable.

Water-soluble biocidal compounds are known. For example, DE 100 43 287 describes antimicrobial polymers with depot action, the depot action being based on a water-soluble oligomer content in the polymers. The water-soluble oligomers are slowly extracted from the polymer, so that biocidal action in solution is observed alongside the contact-microbicidal action. DE 100 43 285 discloses a process which can prepare these water-soluble antimicrobial oligomers.

DE 100 48 613 likewise describes water-soluble antimicrobial oligomers, which have improved microbicidal action through reaction with ketones and/or with aldehydes.

### SUMMARY OF THE INVENTION

One object of the present invention is therefore to provide a method which combines the effects of water-insoluble antimicrobial polymers and biocides which are less environmentally hazardous than the conventional biocides described.

Another object of the invention is to provide a method to ensure that, even in flow-through systems, there is adequate bioavailability of the active agent.

These and other objects have now been attained with the present invention, the first embodiment of which provides an antimicrobial controlled-release composition, which includes at least one antimicrobial polymer prepared from at least one of the following monomers selected from the group including:

2-tert-butylaminoethyl methacrylate, 2-diethylaminoethyl methacrylate, 2-diethylaminomethyl methacrylate, 2-tert-butylaminoethyl acrylate, 3-dimethylaminopropyl acrylate, 2-diethylaminoethyl acrylate, 2-dimethylaminoethyl acrylate, dimethylaminopropylmethacrylamide, diethylaminopropylmethacrylamide, N-3-dimethylaminopropylacrylamide, 2-methacryloyloxyethyltrimethylammonium methosulfate, 2-diethylaminoethyl methacrylate, 2-methacryloyloxyethyltrimethylammonium chloride, 3-methacryloylaminopropyltrimethylammonium chloride, 2-methacryloyloxyethyltrimethylammonium chloride, 2-acryloyloxyethyl-4-benzoyldimethylammonium bromide, 2-methacryloyloxyethyl-4-benzoyldimethylammonium bromide, allyltriphenylphosphonium bromide, allyltriphenylphosphonium chloride, 2-acrylamido-2-methyl-1-propanesulfonic acid, 2-diethylaminoethyl vinyl ether, and 3-aminopropyl vinyl ether, and combinations thereof; and

at least 0.5% by weight of at least one organic solvent.

Another object of the invention provides a process for preparing the above-described composition by free-radical polymerization of one or more of the above-described monomers with incorporation of at least 0.5% by weight of at least one organic solvent.

### BRIEF DESCRIPTION OF THE PREFERRED EMBODIMENTS

Various other objects, features and attendant advantages of the present invention will be more fully appreciated as the same becomes better understood from the following detailed description.

Surprisingly, it has now been found that plasticizing of antimicrobial polymers via addition of organic solvents can give systems which meet the requirements described in an almost ideal manner. Since multiple and automated generation or regeneration of these formulations is possible both during the preparation process and subsequently, and whenever required, e.g. by subsequent impregnation with organic solvents, it is possible by this means to create processes which are equally attractive from an economic and an environmental point of view, and which prepare biocidal controlled-release systems.

Corresponding antimicrobial controlled-release coatings may be obtained directly by dissolving antimicrobial polymers in one or more organic solvents, and then applying the product to a surface, and then in removing the solvents through a subsequent drying process. Preferably, the solvent is removed completely subject to the requirement that at least 0.5% by weight remains.

Not wishing to be bound by theory, it is believed that the added solvent molecules plasticize the antimicrobial polymers in the present invention, and this in combination with the presence of water brings about a carrier effect with respect to these polymers and releases small amounts of antimicrobial polymer into the aqueous phase. This release continues and persists as long as there is still an appropriate concentration of bound organic solvent.

Preferably, the composition and/or antimicrobial polymer of the invention is at least partially water-soluble.

The leaching rate of the antimicrobial systems thus treated is preferably enhanced by the presence of hydrophilic functional groups in the starting molecules. Since the water-soluble polymer fractions are a part of the antimicrobial polymer, this is a direct method of creating a controlled-release formulation of these systems. These fractions may be optionally for specific applications by extracting the antimicrobial polymers with water or with an aqueous solution, and separating off the water-soluble constituents by filtration or dialysis, or in the simplest case decanting the aqueous phase.

The content of organic solvent is generally in the range from 0.5 to 60% by weight, preferably in the range from 0.5 to 30% by weight, in particular in the range from 0.5 to 10% by weight, based on the total weight of antimicrobial polymer and organic solvent and any other additives introduced into the polymer. These ranges include all values and subranges therebetween, including 0.6, 0.7, 0.8, 0.9, 1.0, 5, 7, 9, 15, 25, 35 and 45%.

The plasticizing organic solvent used may be almost any organic solvent which is absorbed by the antimicrobial polymers at more than half of one percent by weight. Examples of these include one or more alcohols, esters, ketones, aldehydes, ethers, acetates, aromatics, hydrocarbons, halogenated hydrocarbons, and organic acids, in particular methanol, ethanol, propanol, butanol, acetone, methyl ethyl ketone, butyl acetate, acetaldehyde, ethylene glycol, propylene glycol, THF, diethyl ether, dioxane, toluene, n-hexane, cyclohexane, cyclohexanol, xylene, DMF, acetic acid, and chloroform. Mixtures are possible.

In addition to the monomers mentioned above, at least one other aliphatically unsaturated monomer may be used during the preparation of the antimicrobial polymers. Preferred monomers for this purpose are acrylic and/or methacrylic compounds, e.g. MMA, or styrene, acrylamides, acrylonitriles, allyl compounds, vinyl ketones, vinyl acetate, vinyl esters, vinyl ethers, vinylacetic acid, or acrylic acid. Combinations are possible.

Molar proportions of the other monomers from 1 to 50 mol %, preferably from 5 to 20 mol %, are possible without any loss of the antimicrobial action of the antimicrobial polymer. These ranges include all values and subranges therebetween, including 2, 3, 4, 5, 6, 7, 8, 9, 10, 25, 35 and 45 mol %.

The term “polymer” used herein is not meant to be limiting, and may include any polymeric and copolymeric structures, for example, random, block, comb, graft, star, etc.

Preferably, the weight-average molecular weight of the antimicrobial polymer ranges from 20,000 to 5,000,000, more preferably from 50,000 to 1,000,000, and most preferably from 100,000 to 500,000. These ranges include all values and subranges therebetween, including 30,000, 75,000, 200,000, 400,000, 800,000, 2,000,000, 3,000,000, and 4,000,000.

The organic solvent may be incorporated by dissolution of the antimicrobial polymer in the organic solvent and removal of the same, e.g. by evaporation or heating. Preferably, the solvent is removed completely subject to the requirement that at least 0.5% by weight remains.

It is also possible to mix the antimicrobial polymer and the solvent in an extruder or kneader.

The present invention also provides the use of the antimicrobial coatings produced according to the invention for producing modified polymer substrates, antimicrobial products, and the resultant products themselves. Products of this type are preferably based on polyamides, polyurethanes, polyether block amides, polyesteramides or -imides, PVC, polyolefins, silicones, polysiloxanes, polymethacrylate, or polyterephthalates, or metals, wood, glass, or ceramics, the surfaces of which have been coated with polymers of the invention.

Preferred examples of antimicrobial products of this type are machine parts for processing food or drink, components of air conditioning systems, coated pipes, semifinished products, roofing, bathroom or toilet items, kitchen items, components of sanitary equipment, components of animal cages or of animal houses, recreational products for children, components of water systems, packaging for food or drink, operating units (touch panels) of devices, and contact lenses.

The coatings of the invention may be used wherever importance is placed on surfaces which are free from bacteria, algae, and fungi, i.e. microbicidal surfaces or surfaces with release properties. Other preferred examples of the use of the coatings of the invention are found in the following sectors:

Marine: boat hulls, docks, buoys, drilling platforms, ballast water tanks

Construction: roofing, basements, walls, facades, greenhouses, sun protection, garden fences, wood protection

Sanitary: public conveniences, bathrooms, shower curtains, toilet items, swimming pool, sauna, jointing, sealing compounds

Food and drink: machines, kitchen, kitchen items, sponges, recreational products for children, packaging for food or drink, milk processing, drinking water systems, cosmetics

Machine parts: air conditioning systems, ion exchangers, process water, solar powered units, heat exchangers, bioreactors, membranes

Medical technology: contact lenses, diapers, membranes, implants

Consumer articles: automobile seats, clothing (socks, sport clothing), hospital equipment, door handles, telephone handsets, public conveyances, animal cages, cash registers, carpeting, wall coverings.

The present invention also provides the use of items for medical technology or hygiene products produced using the controlled-release coatings of the invention or process of the invention. The preferred materials mentioned above are again applicable. Other preferred examples of hygiene products of this type are toothbrushes, toilet seats, combs, and packaging materials. Some hygiene items also include other articles which may come into contact with larger numbers of people, such as telephone handsets, stair rails, door handles, window catches, and grab straps and grab handles in public conveyances. Preferred examples of items for medical technology are catheters, tubing, protective or backing films, and also surgical instruments.

The controlled-release formulations of the invention are also useful as biofouling inhibitors, preferably in cooling circuits. To prevent damage to cooling circuits by infestation with algae or bacteria, the circuits would otherwise have to be cleaned frequently or oversized. In open cooling systems, as usually found in power plants and chemical plants, the addition of microbicidal substances such as formalin is not possible. Accordingly, the invention would be particularly suitable in these applications.

Other microbicidal substances are frequently highly corrosive or form foams, which prevents their use in systems of this type.

In contrast, the controlled-release formulations of the invention or blends of these with other polymers may be fed in finely dispersed form into the process water. The bacteria are killed and, if necessary, removed from the system by filtering off the dispersed polymer/blend. Deposits of bacteria or algae on sections of the plant can thus be effectively prevented.

The present invention also provides a process for sterilizing cooling water streams, by adding the antimicrobial controlled-release formulations of the invention in dispersed form to the cooling water.

The dispersed form of the controlled-release formulations of the invention can be obtained by milling the material, e.g. in a jet mill. The size distribution of the resultant particles when they are used is preferably from 0.001 to 3 mm (particle diameter), firstly providing a large surface for killing the bacteria or algae and secondly enabling, if required, ready separation from the cooling water, e.g. by filtration. This range includes all values and subranges therebetween, including 0.005, 0.01, 0.0, 0.1, 0.5, 1, 1.5, 2 and 2.5 mm.

A preferred embodiment of the process is to continuously remove from the system a proportion (from 5 to 10% by mass or volume) of the controlled-release formulations used and replace it with an appropriate amount of fresh material. As an alternative, a number of microbes in the water may be checked and further antimicrobial controlled-release formulation added as required. Depending on the quality of the water, it is sufficient to use from 0.1 to 100 g of antimicrobial polymer formulation per m³ of cooling water. This range includes all values and subranges therebetween, including 0.5, 0.7, 1, 1.5, 2, 5, 10, 25, 50 and 75 g per m³.

### EXAMPLES

Having generally described this invention, a further understanding can be obtained by reference to certain specific examples which are provided herein for purposes of illustration only and are not intended to be limiting unless otherwise specified.

### Example 1

45 mL of tert-butylaminoethyl methacrylate (Aldrich) and 230 mL of ethanol are charged to a three-necked flask and heated to 65° C. under a stream of argon. 0.4 g of azobisisobutyronitrile dissolved in 20 mL of ethanol is then slowly added dropwise, with stirring. The mixture is heated to 70° C. and stirred at this temperature for 6 hours. After expiration of this time, the solvent is removed from the reaction mixture by distillation. The product is then dried in vacuo at 50° C. for 24 hours.

### Example 1a

The reaction product from Example 1 is ground in a mortar and extracted for 24 hours with 200 mL of water heated to 37° C. The supernatant liquor is then filtered through a 0.2 micrometer pore filter. 2 mL of this solution are shaken with 20 mL of a test microbial suspension of Pseudomonas aeruginosa. After a contact time of 4 hours, 1 mL of the test microbial suspension is removed, and the number of microbes in the test mixture is determined. After expiration of this time, the number of microbes has remained constant at 10⁷ microbes per mL.

### Example 1b

5 g of the product from Example 1 are dissolved in 95 g of cyclohexane. 20 mL of this solution are placed in a glass beaker. The solvent is removed at 35° C. over a period of 48 hours in a drying cabinet, so that a polymer film remains on the base of the glass beaker. This film is extracted for 24 hours with 200 mL of water heated to 37° C. 2 mL of this solution are shaken with 20 mL of a test microbial suspension of Pseudomonas aeruginosa. After a contact time of 4 hours, 1 mL of the test microbial suspension is removed, and the number of microbes in the test mixture is determined. After expiration of this time, the number of microbes has fallen from 10⁷ to 10³ microbes per mL. Over a period of 14 days, the film is extracted as described above, each time for 24 hours, with 200 mL of water heated to 37° C., and the water is then subjected to microbiological testing. All of the determinations show a fall of from 4 to 5 logarithmic levels in the number of microbes.

### Example 1c

5 g of the product from Example 1 are dissolved in 95 g of cyclohexane. 20 mL of this solution are placed in a glass beaker. The solvent is removed over a period of 48 hours at 35° C. in a drying cabinet, so that a polymer film remains on the base of the glass beaker. The film is then dried for 24 hours at 5 mbar at 50° C. in a vacuum drying cabinet. This film is extracted for 24 hours with 200 mL of water heated to 37° C. 2 mL of this solution are shaken with 20 mL of a test microbial suspension of Pseudomonas aeruginosa. After a contact time of 4 hours, 1 mL of the test microbial suspension is removed, and the number of microbes in the test mixture is determined. After expiration of this time, the number of microbes has remained constant at 10⁷ microbes per mL.

### Example 1d

The polymer film from Example 1c is treated with 20 mL of cyclohexane. The solvent is removed over a period of 48 hours at 35° C. in a drying cabinet so that a polymer film remains on the base of the glass beaker. This film is extracted for 24 hours with 200 mL of water heated to 37° C. 2 mL of this solution are shaken with 20 mL of a test microbial suspension of Pseudomonas aeruginosa. After a contact time of 4 hours, 1 mL of the test microbial suspension is removed, and the number of microbes in the test mixture is determined. After expiration of this time, the number of microbes has fallen from 10⁷ to 10³ microbes per mL.

### Example 1e

20 mL of a test microbial suspension of Pseudomonas aeruginosa are shaken with 0.5 mL of cyclohexane. After a contact time of 4 hours, 1 mL of the test microbial suspension is removed, and the number of microbes in the test mixture is determined. After expiration of this time, the number of microbes has remained constant at 10⁷ microbes per mL.

### Example 2

40 mL of dimethylaminopropylmethacrylamide (Aldrich) and 200 mL of ethanol are charged to a three-necked flask and heated to 65° C. under a stream of argon. 0.4 g of azobisisobutyronitrile dissolved in 20 mL of ethanol are then slowly added dropwise, with stirring. The mixture is heated to 70° C. and stirred at this temperature for 6 hours. After expiration of this time, the solvent is removed from the reaction mixture by distillation, and the reaction mixture is dried in vacuo for 24 hours at 50° C. The product is then dissolved in 200 mL of acetone, and then the solvent is removed from the reaction mixture by distillation, and the reaction mixture is dried in vacuo for 24 hours at 50° C. The reaction product is then finely ground in a mortar.

### Example 2a

The reaction product is ground in a mortar and extracted for 24 hours with 200 mL of water heated to 37° C. The supernatant liquor is then filtered through a 0.2 micrometer pore filter. 2 mL of this solution are shaken with 20 mL of a test microbial suspension of Staphylococcus aureus. After a contact time of 4 hours, 1 mL of the test microbial suspension is removed, and the number of microbes in the test mixture is determined. After expiration of this time, the number of microbes has remained constant at 10⁷ microbes per mL.

### Example 2b

5 g of the product from Example 2 are dissolved in 95 g of cyclohexane. 20 mL of this solution are placed in a glass beaker. The solvent is removed at 35° C. over a period of 48 hours in a drying cabinet, so that a polymer film remains on the base of the glass beaker. This film is extracted for 24 hours with 200 mL of water heated to 37° C. 2 mL of this solution are shaken with 20 mL of a test microbial suspension of Staphylococcus aureus. After a contact time of 4 hours, 1 mL of the test microbial suspension is removed, and the number of microbes in the test mixture is determined. After expiration of this time, the number of microbes has fallen from 10⁷ to 10⁴ microbes per mL. Over a period of 14 days, the film is extracted as described above, each time for 24 hours, with 200 mL of water heated to 37° C., and the water is then subjected to microbiological testing. All of the determinations show a fall of from 3 to 4 logarithmic levels in the number of microbes.

### Example 2c

5 g of the product from Example 2 are dissolved in 95 g of cyclohexane. 20 mL of this solution are placed in a glass beaker. The solvent is removed over a period of 48 hours at 35° C. in a drying cabinet, so that a polymer film remains on the base of the glass beaker. The film is then dried for 24 hours at 5 mbar at 50° C. in a vacuum drying cabinet. This film is extracted for 24 hours with 200 mL of water heated to 37° C. 2 mL of this solution are shaken with 20 mL of a test microbial suspension of Staphylococcus aureus. After a contact time of 4 hours, 1 mL of the test microbial suspension is removed, and the number of microbes in the test mixture is determined. After expiration of this time, the number of microbes has remained constant at 10⁷ microbes per mL.

### Example 2d

The polymer film from Example 2c is treated with 20 mL of cyclohexane. The solvent is removed over a period of 48 hours at 35° C. in a drying cabinet so that a polymer film remains on the base of the glass beaker. This film is extracted for 24 hours with 200 mL of water heated to 37° C. 2 mL of this solution are shaken with 20 mL of a test microbial suspension of Staphylococcus aureus. After a contact time of 4 hours, 1 mL of the test microbial suspension is removed, and the number of microbes in the test mixture is determined. After expiration of this time, the number of microbes has fallen from 10⁷ to 10³ microbes per mL.

### Example 3

45 mL of tert-butylaminoethyl methacrylate (Aldrich) and 230 mL of ethanol are charged to a three-necked flask and heated to 65° C. under a stream of argon. 0.4 g of azobisisobutyronitrile dissolved in 20 mL of ethanol is then slowly added dropwise, with stirring. The mixture is heated to 70° C. and stirred at this temperature for 6 hours. After expiration of this time, the solvent is removed from the reaction mixture by distillation. The product is then dried in vacuo at 50° C. for 24 hours.

### Example 3a

The reaction product from Example 3 is ground in a mortar and extracted for 24 hours with 200 mL of water heated to 37° C. 2 mL of this solution are shaken with 20 mL of a test microbial suspension of Staphylococcus aureus. After a contact time of 4 hours, 1 mL of the test microbial suspension is removed, and the number of microbes in the test mixture is determined. After expiration of this time, the number of microbes has remained constant at 10⁷ microbes per mL.

### Example 3b

5 g of the product from Example 3 are dissolved in 95 g of ethanol. 20 mL of this solution are placed in a glass beaker. The solvent is removed at 35° C. over a period of 48 hours in a drying cabinet, so that a polymer film remains on the base of the glass beaker. This film is extracted for 24 hours with 200 mL of water heated to 37° C. 2 mL of this solution are shaken with 20 mL of a test microbial suspension of Staphylococcus aureus. After a contact time of 4 hours, 1 mL of the test microbial suspension is removed, and the number of microbes in the test mixture is determined. After expiration of this time, the number of microbes has fallen from 10⁷ to 10³ microbes per mL. Over a period of 14 days, the film is extracted as described above, each time for 24 hours, with 200 mL of water heated to 37° C., and the water is then subjected to microbiological testing. All of the determinations show a fall of from 3 to 4 logarithmic levels in the number of microbes.

### Example 3c

5 g of the product from Example 3 are dissolved in 95 g of ethanol. 20 mL of this solution are placed in a glass beaker. The solvent is removed over a period of 48 hours at 35° C. in a drying cabinet, so that a polymer film remains on the base of the glass beaker. The film is then dried for 24 hours at 5 mbar at 50° C. in a vacuum drying cabinet. This film is extracted for 24 hours with 200 mL of water heated to 37° C. 2 mL of this solution are shaken with 20 mL of a test microbial suspension of Staphylococcus aureus. After a contact time of 4 hours, 1 mL of the test microbial suspension is removed, and the number of microbes in the test mixture is determined. After expiration of this time, the number of microbes has remained constant at 10⁷ microbes per mL.

### Example 3d

The polymer film from Example 3c is treated with 20 mL of ethanol. The solvent is removed over a period of 48 hours at 35° C. in a drying cabinet so that a polymer film remains on the base of the glass beaker. This film is extracted for 24 hours with 200 mL of water heated to 37° C. 2 mL of this solution are shaken with 20 mL of a test microbial suspension of Staphylococcus aureus. After a contact time of 4 hours, 1 mL of the test microbial suspension is removed, and the number of microbes in the test mixture is determined. After expiration of this time, the number of microbes has fallen from 10⁷ to 10⁴ microbes per mL.

### Example 3e

20 mL of a test microbial suspension of Staphylococcus aureus are shaken with 0.5 mL of ethanol. After a contact time of 4 hours, 1 mL of the test microbial suspension is removed, and the number of microbes in the test mixture is determined. After expiration of this time, the number of microbes has remained constant at 10⁷ microbes per mL.

Obviously, numerous modifications and variations of the present invention are possible in light of the above teachings. It is therefore to be understood that within the scope of the appended claims, the invention may be practiced otherwise than as specifically described herein.

This application is based on German patent application DE 10145529.1, filed Sep. 14, 2001, the entire contents of which are hereby incorporated by reference.

1. An antimicrobial, controlled-release composition, comprising:
at least one antimicrobial polymer comprising at least one polymerized monomer unit selected from the group consisting of:
2-tert-butylaminoethyl methacrylate, 2-diethylaminoethyl methacrylate, 2-diethylaminomethyl methacrylate, 2-tert-butylaminoethyl acrylate, 3-dimethylaminopropyl acrylate, 2-diethylaminoethyl acrylate, 2-dimethylaminoethyl acrylate, dimethylaminopropylmethacrylamide, diethylaminopropylmethacrylamide, N-3-dimethylaminopropylacrylamide, 2-methacryloyloxyethyltrimethylammonium methosulfate, 2-diethylaminoethyl methacrylate, 2-methacryloyloxyethyltrimethylammonium chloride, 3-methacryloylaminopropyltrimethylammonium chloride, 2-methacryloyloxyethyltrimethylammonium chloride, 2-acryloyloxyethyl-4-benzoyldimethylammonium bromide, 2-methacryloyloxyethyl-4-benzoyldimethylammonium bromide, allyltriphenylphosphonium bromide, allyltriphenylphosphonium chloride, 2-acrylamido-2-methyl-1-propanesulfonic acid, 2-diethylaminoethyl vinyl ether, 3-aminopropyl vinyl ether, and combinations thereof; and
at least 0.5% by weight of at least one organic solvent.

2. The composition as claimed in claim 1, wherein the organic solvent is selected from the group consisting of an alcohol, an ester, a ketone, an aldehyde, an ether, an acetate, an aromatic, a hydrocarbon, a halogenated hydrocarbon, an organic acid, and a mixture thereof.

3. The composition as claimed in claim 1, wherein the organic solvent is selected from the group consisting of methanol, ethanol, propanol, butanol, acetone, methyl ethyl ketone, butyl acetate, acetaldehyde, ethylene glycol, propylene glycol, THF, diethyl ether, dioxane, toluene, n-hexane, cyclohexane, cyclohexanol, xylene, DMF, acetic acid, chloroform, and a mixture thereof.

4. The composition as claimed in claim 1, wherein said polymer further comprises at least one polymerized aliphatically unsaturated monomer.

5. The composition as claimed in claim 4, wherein the aliphatically unsaturated monomer comprises acrylic acid, methacrylic acid, or a combination thereof.

6. A process for preparing an antimicrobial controlled-release composition, comprising
free-radical polymerization of at least one monomer unit selected from the group consisting of 2-tert-butylaminoethyl methacrylate, 2-diethylamino ethyl methacrylate, 2-diethylaminomethyl methacrylate, 2-tert-butylaminoethyl acrylate, 3-dimethylaminopropyl acrylate, 2-diethylaminoethyl acrylate, 2-dimethylaminoethyl acrylate, dimethylaminopropylmethacrylamide, diethylaminopropylmethacrylamide, N-3-dimethylaminopropylacrylamide, 2-methacryloyloxyethyltrimethylammonium methosulfate, 2-diethylaminoethyl methacrylate, 2-methacryloyloxyethyltrimethylammonium chloride, 3-methacryloylaminopropyltrimethylammonium chloride, 2-methacryloyloxyethyltrimethylammonium chloride, 2-acryloyloxyethyl-4-benzoyldimethylammonium bromide, 2-methacryloyloxyethyl-4-benzoyldimethylammonium bromide, allyltriphenylphosphonium bromide, allyltriphenylphosphonium chloride, 2-acrylamido-2-methyl-1-propanesulfonic acid, 2-diethylaminoethyl vinyl ether, 3-aminopropyl vinyl ether, and combinations thereof, to form a polymer; and
contacting said polymer with at least 0.5% by weight of at least one organic solvent.

7. The process as claimed in claim 6, further comprising, prior to contacting the polymer and the solvent, removing water-soluble constituents of the polymer by extracting the polymer with water or with an aqueous solution, and then separating off the aqueous phase.

8. The process as claimed in claim 6, wherein contacting the polymer and the solvent comprises dissolving the polymer in the organic solvent.

9. The process as claimed in claim 6, wherein contacting the polymer and the solvent takes place in an extruder or kneader.

10. The process as claimed in claim 6, wherein said polymerization further comprises polymerizing at least one other aliphatic unsaturated monomer with the monomer units.

11. The process as claimed in claim 10, wherein the other aliphatically unsaturated monomer comprises acrylic acid, methacrylic acid, or both.

12. A coated product, comprising a surface coated with the composition as claimed in claim 1.

13. A surface coating or protective paint, comprising the composition of claim 1.

14. A process for removing microbes from cooling water streams, which comprises contacting said cooling water with the composition of claim 1.

15. A process for imparting antimicrobial activity to a surface, comprising coating said surface with the composition as claimed in claim 1.

16. The composition as claimed in claim 1, wherein said polymer has a weight-average molecular weight ranging from 20,000 to 5,000,000.

17. The composition as claimed in claim 1, wherein said polymer has a weight-average molecular weight ranging from 50,000 to 1,000,000.
